(19) United States
(12) Patent Application Publication
(10) Pub. No.: US 2007/0179410 A1
(43) Pub. Date: Aug. 2, 2007

(54) Cervigage

(76) Inventors: Ajay Mahajan, Murphyboro, IL (US); Bradley Schwartz, Springfield, IL (US); Leanne Schwartz, Murphysboro, IL (US)

Correspondence Address: Wilson Daniel Swayze, Jr, 3804 Clearwater Ct., Plano, TX, 75025, US

(21) Appl. No.: 11/450,182

(22) Filed: Jun. 9, 2006

Related U.S. Application Data
(60) Provisional application No. 60/763,289, filed on Jan. 30, 2006.

(51) Int. Cl. A61B 5/103

(52) U.S. Cl. 600/591; 600/588

(57) ABSTRACT

A measurement device for measuring the diameter and of a cervical os, including a first connector for connecting to a first portion of the cervical os, a second connector for connecting to a second portion of the cervical os, an expandable device positioned between the first connector and the second connector, and the expendable device being expandable to measure the movement of the cervical os.

### PRIORITY

The present invention claims priority under 35 USC section 119 based on a provisional application with a Ser. No. 60/763,289 filed on Jan. 30, 2006.

### FIELD OF THE INVENTION

The present invention relates generally to the field of medical devices and measurements. More particularly, the present invention relates to a method and apparatus for measuring the cervical os diameter of a patient during pregnancy and labor.

### BACKGROUND OF THE INVENTION

U.S. Pat. No. 6,039,701 discloses cervimeters and methods for measuring and detecting changes in a patient's cervix and cervical OS which includes a loop element which is securable to a patient's cervix where a peripheral dimension of the loop varies directly with changes in the cervical diameter.

The uterine cervical canal serves as a conduit for the human fetus to exit the uterus, and the human fetus is subsequently passed through the vagina during the birthing process. It has long been known that the cervix normally undergoes a series of physical and biochemical changes during the latter portions of pregnancy which enhances the ease and safety of the birthing process for the mother and infant. For example, in the early stages of labor, the tissues of the cervical canal soften and become more pliable, the cervix shortens and the diameter of the cervical canal begins to increase at its proximal end of the internal os. As labor progresses, the cervical diameter growth propagates to the distal end of the cervical canal to the external os. In the final stages of labor, the external os dilates allowing for the unobstructed passage of the fetus.

The monitoring of cervical diameter via serial vaginal exams is important in labor-management. This information regarding the progress of labor is used to diagnose such conditions as dysfunctional or arrested labor and cephalopelvic disproportion, determine the necessity for labor augmentation or cesarean section, indicate when the patient should begin to push the baby out and determine the appropriateness of timing of administrating various anesthetic/analegesic agents, among other things. The vaginal exam is performed by inserting the first and second fingers of a gloved hand into the vagina and up to the cervix. By spreading the two fingers across what is perceived to be the internal diameter of the cervix, an assessment is made based on experience, about the cervical dilation in centimeters.

Acceptable, repeatable results can be achieved using this method. However, the variation between measurements can be significant due to the subjective nature of the measurements and may change with different examiners. Such measurements are discrete and must be performed serially to assess labor progress and ascertained whether intervention is required. This procedure carries with it a risk of infections; consequently it is desirable to keep the number of these procedures to a minimum.

It is not surprising that there has been numerous historical attempts to provide a more accurate user-independent cervical diameter measuring device and to provide an automatic cervical diameter measuring device.

### SUMMARY OF THE INVENTION

It is an objective of the invention to provide a measurement device to measure the dilation of the os with a simple and disposable device.

It is an objective of the invention to provide a measurement device that is biocompatible.

It is a further objective of the present invention to convert the output of the measurement device to a digital form and to transmit the output of the measurement device to a centralized monitoring station.

It is a further objective of the present invention to activate an alarm if the output of the measurement device exceeds a predetermined limit.

The measurement device of the present invention should be easy to insert.

The measurement device of the present invention is both safe for the mother and child and comfortable for the mother while in use.

The measurement device of the present invention includes cylinders which are designed to slip out of the mother before the dilation of the mother reaches full effacement, for example 10 cm.

The measurement device of the present invention is comfortable because the measurement device has a small profile, resulting in no discomfort to the mother.

The measurement device of the present invention is flexible and does not cause any trauma to the cervix of the mother or to the baby.

The measurement device of the present invention should accurately determine the cervical diameter.

The measurement device of the present invention should reduce the risk of infection.

The measurement device of the present invention should result in the delivery process for women being less traumatic both physically and emotionally.

The present invention includes a measurement device or mechanical sensor that can measure the cervical os diameter as it widens during pregnancy. As the os thins in the late stages of labor (effacement), the measuring device should fall out or out of the cervix, indicating imminent delivery. The measurement device includes a wire or tape that extends from the measurement device and extends from the body of the woman so that the user of the measuring device can detect changes in the length of the wire or tape.

The measurement device includes a first connector and second connector to be attached to the diametrically opposite ends of the cervical os. Consequently, as the ends of the cervical os separate, the tape or wire is pulled by the separation of the cervical os. By the connection of the first connector and the second connector to the diametrically opposed ends of the cervical os, a direct measurement of the diameter of the cervical os can be obtained. The first and second connector can be connected with a spring to urge the first and second connectors apart. The tape or wire or can be connected to a distal end of the measurement device.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates the measurement device positioned within the cervical os;

FIG. 2 illustrates a cross-sectional view of the measurement device;

FIG. 3 illustrates a cross-sectional view of another measurement device;

FIG. 4 illustrates a perspective view of the measurement device;

FIG. 5 illustrates another cross-sectional view of the measurement device.

### DETAILED DESCRIPTION

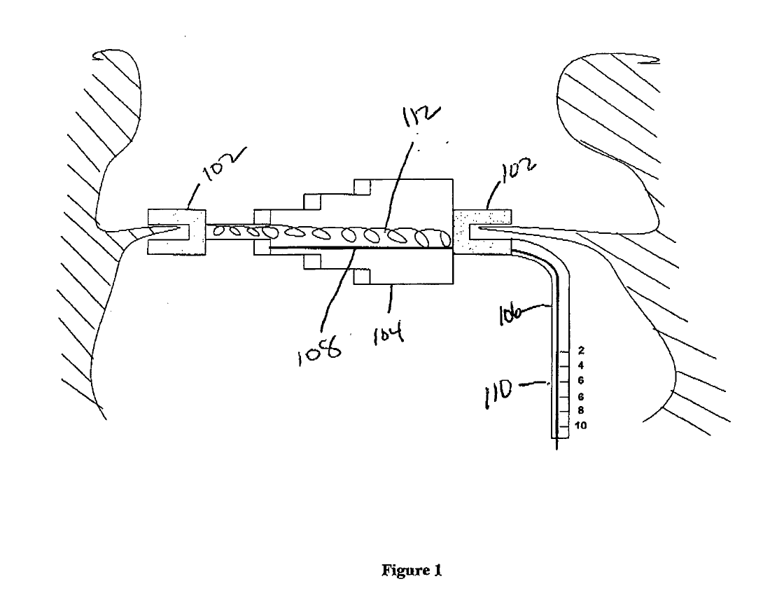

FIG. 1 illustrates the measurement device 100 positioned within the cervical, and the measurement device 100 includes an expandable device 104 which includes a hollow housing to house a spring 112 or other such elastic device connected along a longitudinal axis of the expandable device 104. The expandable device 104 is movable in the longitudinal direction and includes a first connector 102 positioned at one end of the expandable device 104 and a second connector 102 at the other end of the expandable device 104. The first connector 104 and the second connector 104 are connected to diametrically opposing sides of the cervical os, and as a result as the cervical os expands or contracts, the expandable device 104 expands or contracts with the cervical os. The spring 112 traverses the interior of the expandable device 104 and is connected to the expandable device 104 at the distal end and at the proximal end of the expandable device 104. The wire 108 enters the expandable device 104 at the proximal end of the expandable device 104 and is connected to the distal end of the expandable device 104 and is sufficiently flexible to allow the wire 108 to be bent so that it can exit the vagina without discomfort and sufficiently rigid to allow accurate measurement of the diameter of the cervical os.

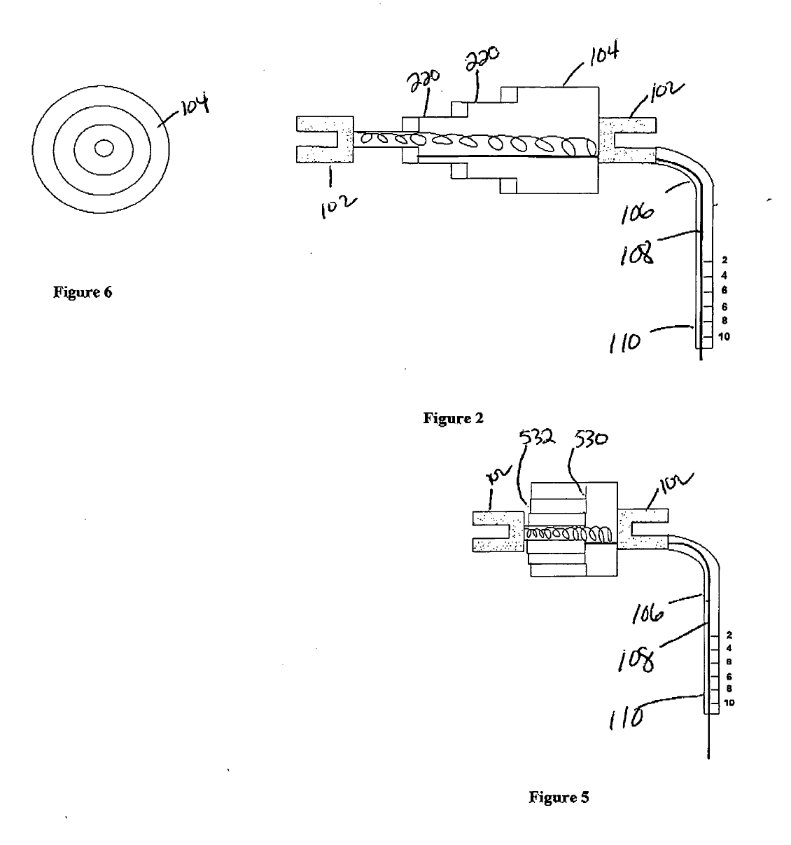

FIG. 2 illustrates that the expandable device 104 includes hollow cylinders 220, where each successive cylinder 220 has a reduced diameter with respect to the preceding cylinder 220 so that the cylinder 220 can move within the interior of the adjacent cylinder 220. As a consequence, the expandable device 104 expands in a telescoping manner by each cylinder 220 moving in the interior of the adjacent cylinder 220.

A cylinder 220 as illustrated in FIG. 5 includes an outward projecting element 532 and an inward projecting element 530 to limit the amount of movement for the cylinders 220. The cylinders 220 are designed to slip out before the dilation of the mother reaches full effacement which is approximately 10 cm. This aspect adds to the safety of the measurement device 100 because the measurement device then can be removed from the mother safely before it can interfere with the delivery of the baby. The measurement device 100 is expelled from the cervix of the mother if the cervix diameter dilates to approximately 10 cm or the head of the baby pushes the measurement device 100 out. Additionally, the measurement device is flexible so that no trauma results to the cervix or the baby.

FIG. 6 illustrates an end view of the expandable device 104

As the cervical os diameter expands, the spring 112 urges first connector 102 and the second connector 102 outwards, and the wire 108 which is connected to the distal end of the expandable device 104 moves towards this distal end of the expandable device 104 as the cervical os diameter expands. The wire 108 is covered by a sheath 106 which is connected to the expandable device 104 to protect the wire 108 and to avoid discomfort. The wire 108 and the sheath 106 exit the vagina and the wire 108 is connected to, in one embodiment, a scale device 110 to allow the user to measure the diameter of the cervical os. The scale device 110 includes indicia such as the number of inches or millimeters on a scale so that the user can conveniently monitor the diameter of the cervical os by the movement of the wire 108. As the cervical os expands, the connectors 102 will move with the cervical os, and as the connectors 102 move, the expandable device 104 will expand along the diameter of the cervical os. The spring 112 will urge the connectors 102 outwards, and the wire 108 will move towards the distal end of the expandable device 104. Consequently, the end of the wire 108 connected to the scale device 110 will move towards the vagina and the user can accurately determine the amount of movement of the cervical os diameter by examining the indicia of the scale device 110.

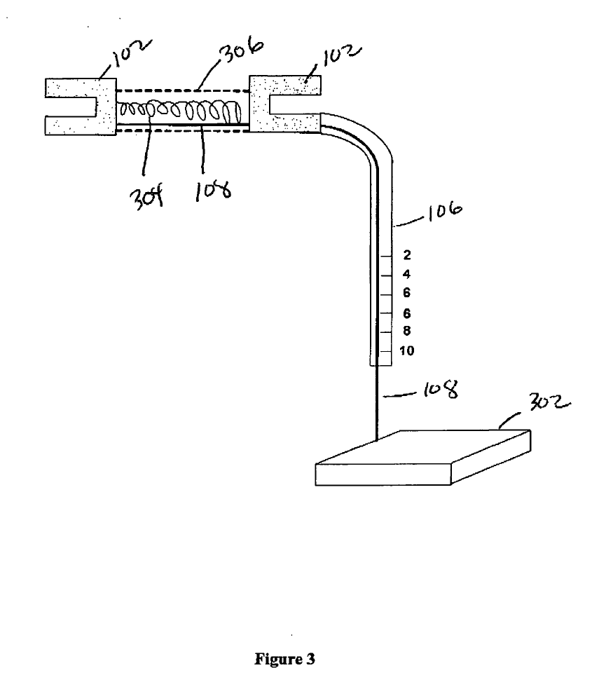

FIG. 3 illustrates another embodiment of the expandable device of the present invention which includes a weakened support connector 306 to support the connectors 102 in a spaced relationship. The weakened support connector 306 may separate after the connectors 102 are placed on the cervical os.

The expandable device 104 should fall off or fall out of the cervix prior to delivery. The sheath 106 may be formed from transparent material and may include indicia so that the movement of the wire 108 can be observed by the user. The measurement device 100 may be disposable, 100% inert and biocompatible with no risk to baby or mother. The monitoring device 402 converts the length of travel of the wire 108 to a digital number which is read out, can transmit this digital number and provide audible or visual alarms. The expandable device 104 could be made from flexible plastic, and the connectors 102 could be formed from silicon.

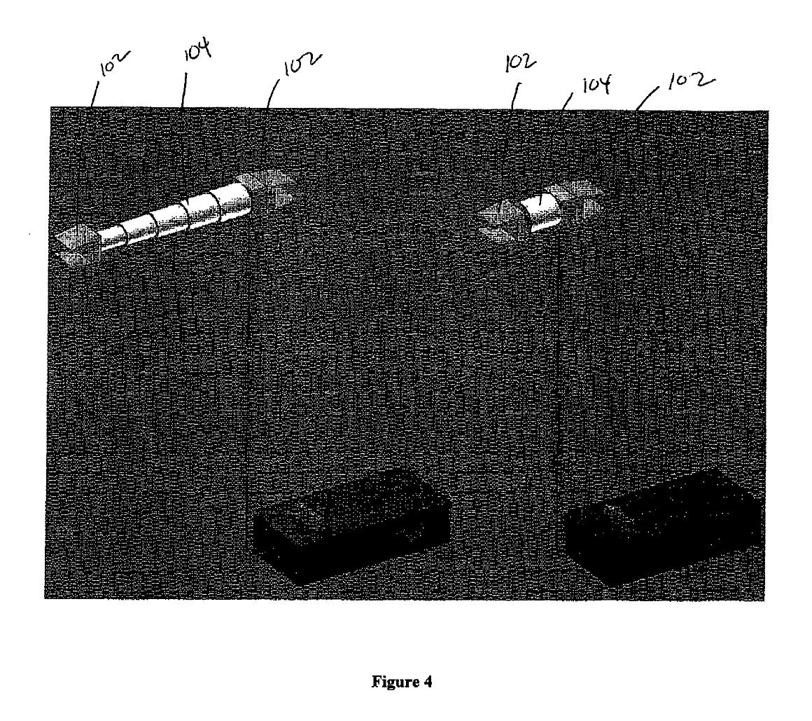

FIG. 4 shows a perspective view of the measuring device 400 including a monitoring device 402 to perform various functions associated with the measuring device 400. For example, the monitoring device 402 can record changes in the movement of the wire 108 as a function of time so that the user can view these changes to determine if the delivery is proceeding satisfactorily. The monitoring device 400 could perform an integration to determine the rate of change of movement of the wire 108 and could activate an alarm if the movement of the wire 108 exceeds a predetermined threshold. The monitoring device 402 could communicate with other remote devices such as the personal computer a cell phone or other suitable device at a remote location in order to keep the user advised of the movement of the cervical os. The communication could be through a wired connection, a wireless connection, the Internet or any other suitable communication device.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A measurement device for measuring the diameter of a cervical os, comprising:
a first connector for connecting to a first portion of said cervical os;
a second connector for connecting to a second portion of said cervical os;
an expandable device positioned between said first connector and said second connector;
said expandable device being expandable to measure the diameter of said cervical os.

2. A measurement device for measuring the diameter of a cervical os as in claim 1, wherein said measurement device includes a spring to urge said first and second connectors outward towards said cervical os.

3. A measurement device for measuring the diameter of a cervical os as in claim 1, wherein said measurement device includes a wire to measure the movement of said cervical os.

4. A measurement device for measuring the diameter of a cervical os as in claim 3, wherein said measurement device includes a sheath to cover said wire.

5. A measurement device for measuring the diameter of a cervical os as in claim 4, wherein said sheath is transparent.

6. A measurement device for measuring the diameter of a cervical os as in claim 5, wherein said sheath includes indicia to measure said diameter of said cervical os.

7. A measurement device for measuring the diameter of a cervical os as in claim 1, wherein said measuring device includes a monitoring device to monitor said movement of said cervical os.

8. A measurement device for measuring the diameter of the cervical os as in claim 7, wherein said measurement device is safely expelled before the diameter reaches full effacement.

9. A measurement device for measuring the diameter of a cervical os as in claim 7, wherein said monitoring device is wirelessly connected to a remote device.

10. A method for measuring the diameter of a cervical os using a measuring device, comprising the steps of:
connecting to a first portion of said cervical os with a first connector;
connecting to a second portion of said cervical os with a second connector;
positioning an expandable device between said first connector and said second connector;
expanding said expendable device to measure the diameter of said cervical os.

11. A method for measuring the diameter of a cervical os using a measuring device as in claim 10, wherein said method includes the step of urging said first and second connectors outward towards said cervical os.

12. A method for measuring the diameter of a cervical os using a measuring device as in claim 10, wherein said method includes the step of measuring the diameter of said cervical os with a wire.

13. A method for measuring the diameter of a cervical os using a measuring device as in claim 12, wherein said method includes the step of forming a sheath to cover said wire.

14. A method for measuring the diameter of a cervical os using a measuring device as in claim 13, wherein said sheath is transparent.

15. A method for measuring the diameter of a cervical os using a measuring device as in claim 14, wherein the method includes the step of forming indicia on said sheath to measure said diameter of said cervical os.

16. A method for measuring the diameter of a cervical os using a measuring device as in claim 10, wherein said method includes the step of monitoring said diameter of said cervical os with a monitoring device.

17. A method for measuring the diameter of a cervical os using a measuring device as in claim 16, wherein said monitoring device is connected to a remote device.

18. A method for measuring the diameter of a cervical os using a measuring device as in claim 16, wherein said monitoring device is wirelessly connected to a remote device.

19. A measurement device for measuring the diameter of the cervical os as in claim 7, wherein said measurement device can be comfortably used for a long period of time.
